(19) United States
(12) Patent Application Publication
(10) Pub. No.: US 2026/0364349 A1
(43) Pub. Date: Nov. 19, 2026

(54) Opthalmic Stimulation Device

(71) Applicant: PAUL ERIC JACOBSEN, Sebastian, FL (US)

(72) Inventor: PAUL ERIC JACOBSEN, Sebastian, FL (US)

(21) Appl. No.: 19/210,012

(22) Filed: May 16, 2025

(51) Int. Cl. A61N 1/36; A61N 1/04; A61N 5/06

(52) U.S. Cl. CPC A61N 1/36046; A61N 1/0456; A61N 1/0484; A61N 5/0622; A61N 2005/0626; A61N 2005/0648; A61N 2005/0652; A61N 2005/0659; A61N 2005/0663; A61N 2005/0666

(57) ABSTRACT

An ophthalmic stimulation device for treating eye diseases is described. The ophthalmic stimulation device includes a frame, a strap element coupled to end pieces of the frame, a plurality of electrodes fixedly coupled to the frame, a plurality of light emitters coupled to the frame, and a controller configured to control the operation of the plurality of electrodes and the plurality of light emitters. The electrodes are positioned to engage at orbital positions around the eye of a wearer, and configured to emit electrical microcurrents to stimulate the eye of the wearer. The light emitters emit non-coherent light between 560 and 900 nm and configured to irradiate the eye of the wearer.

### BACKGROUND

### Technical Field

The present disclosure relates to ophthalmic stimulation devices. More particularly, the present disclosure relates to multi-modal ophthalmic stimulation devices.

### Related Art

Macular degeneration is an eye condition which affects the macula of the retina. Consequently, this affects a person's eyesight at the center of their field of vision. In other words, a person suffering from macular degeneration may not be able to see clearly directly in front of them. Macular degeneration is commonly age related (Age related Macular Degeneration, or simply AMD), and usually affects people above the age of 50. In some cases, macular degeneration may also occur in younger people, for example, if they have a family history (i.e. genetic or hereditary) of macular degeneration, or if they are obese, or if they suffer from hypertension or diabetes, or they have lifestyle parameters, including smoking and a diet high in saturated fats.

AMD is progressive, and may first cause blurry central vision, and if left untreated, may progress to completely failed central vision. While there is no real cure for AMD, there are treatments that may help manage the disease, and slow down, arrest, or even reverse the progression of AMD. Some treatments include medications and nutritional supplements to correct deficiencies. AMD may be managed with nutritional supplements such as Vitamin C, Vitamin E, minerals such as Zinc and Copper, carotenoid supplements such as Lutein and Zeaxanthin. AMD may be treated or managed with medications as well.

Other forms of treatment include photodynamic therapy (PDT), which includes injecting a light sensitive drug into the eye, and subsequent laser irradiation, which destroy the extra blood vessels causing the macular degeneration. Alternatively, laser irradiation may also be used in a photocoagulation therapy, to seal and destroy leaky blood vessels in the eye, which were causing the macular degeneration. However, such treatments are in-clinic treatments, requiring expert treatment administration. Patients are required to travel to the clinics for treatments. Such treatments are often also expensive. The treatment devices are also prohibitively expensive and require deep expertise for dosing and treatment administration, thus preventing personal use.

Other treatments for macular degeneration are also described in the art. For example, prosthetic implants or contact lenses to stimulate the eye, based on an external stimulus. For example, photo electrical implants and contact lenses, which generate electrical impulses based on an external light source, to electrically stimulate the eye. Some other photo electrical implants and contact lenses generate infrared energy based on an external wireless electrical source. However, these solutions also suffer from limitations. Implants and prosthesis require an in-clinic procedure or surgery, which often has clinical risks, an associated recovery time, and involve significant post operative care. On the other hand, while photo electrical contact lenses may possibly enable personal use, they are often very expensive. Further, correctly handling and placing the contact lenses also requires good vision, which the patient is already lacking-thus rendering lenses only useful for early onset cases. Additionally, AMD is a disease of the elderly who also may lack the manual dexterity to manipulate contact lenses.

Yet other treatments for macular degeneration described in the art include electrically stimulating the eye externally. Such devices known in the art suffer from various limitations, such as sub-optimal electrode placement, for example at the nose bridge, and temples. Such devices are targeted at muscle stimulation, rather than nerve stimulation, and therefore have only limited or no efficacy for macular degeneration.

Still other treatments known in the art, for improving vision and treating vision disorders include low level photo biomodulation therapy. Photo biomodulation therapies involve red and near infrared (NIR) light and are typically targeted at improving mitochondrial function of the irradiated cells.

There is also an inherent technical conflict in known single mode therapy devices—electrical stimulation solutions are only designed for optimal electrical contact, but ignore optical alignment, while photo stimulation devices are only designed for optimal optical alignment, but have no requirement for correct contact pressure, or for the correct placement of electrodes in a convenient and efficacious manner which focusses the electrical stimulation effects on the macula.

Therefore, there is a need for a device, to address the aforementioned problems of ease of use, treatment efficacy, accurate treatment dosing, and other limitations of the solutions known in the art.

### SUMMARY

Particular embodiments of the present disclosure are described herein below with reference to the accompanying drawings. However, it shall be appreciated that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to employ the present disclosure in virtually any appropriately detailed structure.

The present disclosure describes a novel wearable device for administering multi-modal treatment to the eyes of the wearer for eye diseases. Embodiments described herein describe a combination of low-level, non-coherent photo stimulation and a low-level electrical stimulation, both of which are eye safe for regular use, in a wearable device, particularly convenient for people with weak or impaired vision, and limited manual dexterity.

One embodiment of an ophthalmic stimulation device comprises a frame, a strap element coupled to end pieces of the frame, a plurality of electrodes fixedly coupled to the frame, a plurality of light emitters coupled to the frame, and a controller configured to control the operation of the plurality of electrodes and the plurality of light emitters.

Another embodiment of an ophthalmic stimulation device comprises a frame, an eyepiece coupled to the frame, a strap element coupled to end pieces of the frame, a plurality of electrodes fixedly coupled to the frame, a plurality of light emitters coupled to the eyepiece, and a controller configured to control the operation of the plurality of electrodes and the plurality of light emitters.

The plurality of electrodes are positioned to engage at orbital positions around an eye of a wearer, wherein the plurality of electrodes are configured to emit electrical microcurrents to stimulate the eye of the wearer.

The light emitters emit non-coherent light between 560 and 900 nm, wherein the plurality of light emitters are configured to irradiate the eye of the wearer, with a particular focus on the macula.

### BRIEF DESCRIPTION OF THE DRAWINGS

FIG. 1 illustrates an ophthalmic stimulation device, according to one exemplary embodiment;

FIGS. 2A and 2B illustrate a rear view of an ophthalmic stimulation device, according to one exemplary embodiment;

FIG. 3 illustrates a rear view of an ophthalmic stimulation device, according to one exemplary embodiment;

FIG. 4 illustrates a rear view of an ophthalmic stimulation device, according to one exemplary embodiment;

FIG. 5 illustrates an electrode positioning of the ophthalmic stimulation device, on the face of the wearer, according to various exemplary embodiments; and

FIG. 6 illustrates a block diagram of an ophthalmic stimulation device (OSD) controller, according to one exemplary embodiment.

### DETAILED DESCRIPTION

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

Embodiments of the personal care device described herein provide several advantages over solutions known in the art. Embodiments presented herein describe an Ophthalmic Stimulation Device (OSD) for treating eye and vision disorders and diseases. The OSD is an integrated multi-modal treatment device combining low-level photo stimulation and low-level electrical stimulation therapies for treating eye disorders. The OSD is a wearable device that addresses the challenges of combining photo stimulation and electrical stimulation in a wearable device. Particularly, a substantially rigid frame allows for accurate and consistent aiming of the photo stimulation to the macula/retina. An adjustable/elastic strap element provides the optimal tension so that the electrodes maintain the correct contact pressure against the wearer's skin. Individually extending/retracting electrodes further ensure optimal contact against the wearer's skin for a wide range of face structures, particularly since the frame may be substantially rigid, with minimal to no flexure under normal usage conditions. Embodiments presented herein overcome various challenges associated with effectively combining photo stimulation and electrical stimulation therapy in a single device. Advantageously, such an integrated device enables ease of use and convenience, in that only a single device is required, and multiple devices do not need to be separately controlled or maintained. In particular, the OSD provides optimum contact pressure of the electrodes transmitting the electrical stimulation signals on the wearer's skin, without relying on adhesive gels, or separate electrical stimulation electrode patches that need to be applied on the wearer's face, or on the therapy eyewear or headwear. Consequently, the OSD described herein provides wearer comfort, convenience of use, and ease of maintenance that are absent from devices known in the art—in that a separate electrode patch does not need to be attached, applied, or otherwise handled or maintained. People suffering from AMD are usually older in age, already having impaired vision and limited manual dexterity, and benefit particularly from not having to use separate electrode patches.

The OSD further employs electrical electrodes at the most efficacious location—i.e. the orbital portion of the eye, unlike solutions known in the art, which have sub-optimal electrode placement, such as nose bridge, temples, and so forth. The OSD device may also have an integrated controller which controls the photo stimulation and electrical stimulation, which may be programmable for different treatment courses, and which may also include a communication module to communicate with a remote system, such as at a healthcare provider's location.

While some advantages have been described above and through the remainder of the present disclosure, it shall be appreciated that these advantages are merely exemplary and the OSD described herein may have other advantages.

The present specification make reference to components being “fixedly” couple or attached to one another. The term “fixedly” coupled or attached as used throughout the present specification refers to components being attached to one another, in a manner that they are not separable under normal use of the OSD, for therapeutic ophthalmic stimulation, or for programming, or for charging. In other words, “fixedly coupled” or “fixedly attached” components may either be integrally formed, bonded to one another and therefore not separable, or only be separable for repairs using appropriate tools and techniques. Components that are “fixedly coupled” or “fixedly attached” may or may not be rigidly coupled. In other words, “fixed” coupling also includes flexible, movable, or compressible coupling, while still not being separable under normal therapeutic use.

Turning now to the figures, various embodiments of an ophthalmic stimulation device (hereinafter, OSD) are described herein.

### Opthalmic Stimulation Device

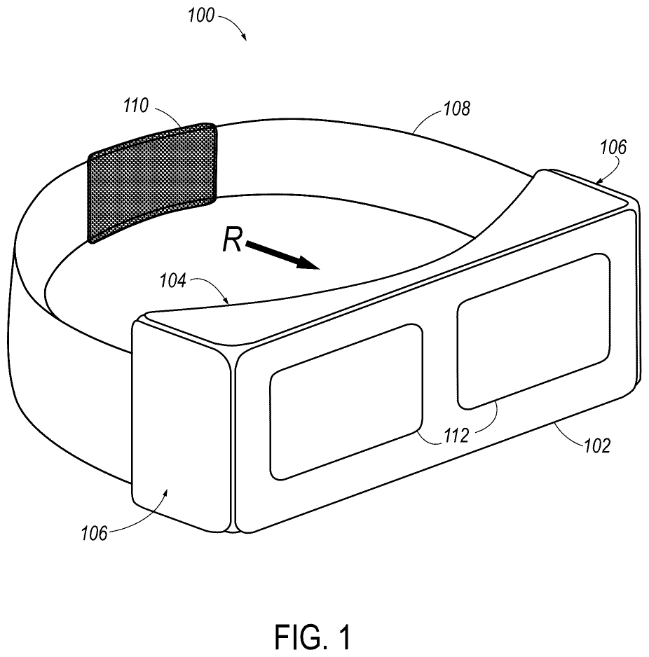

FIG. 1 illustrates an OSD 100, according to one exemplary embodiment. The OSD 100 includes a frame 102, a skin engaging surface 104, endpieces 106, a strap element 108, and an adjustment mechanism 110, and eyepieces 112. The frame 102 functions as the base structure of the OSD 100. The frame 102 may be made of a polymer such as Acrylonitrile butadiene styrene (ABS), polycarbonate, Polylactic acid (PLA), and so forth. Other materials may also be used for the frame 102. The material of the frame 102 may be selected based on parameters such as light weight, and impact resistance, scratch resistance, and so forth-which improve comfort during use, durability during normal use, and ease up maintenance and cleaning.

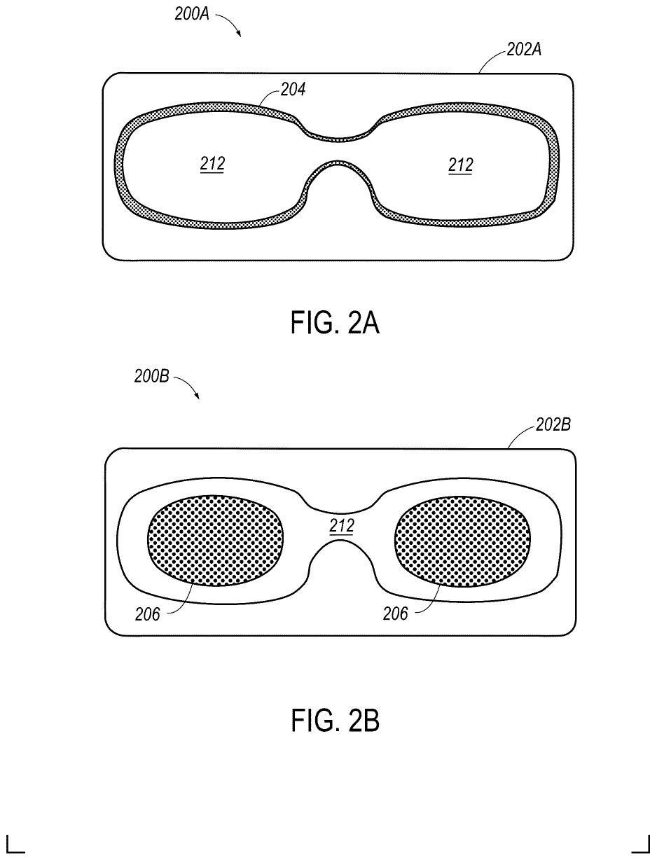

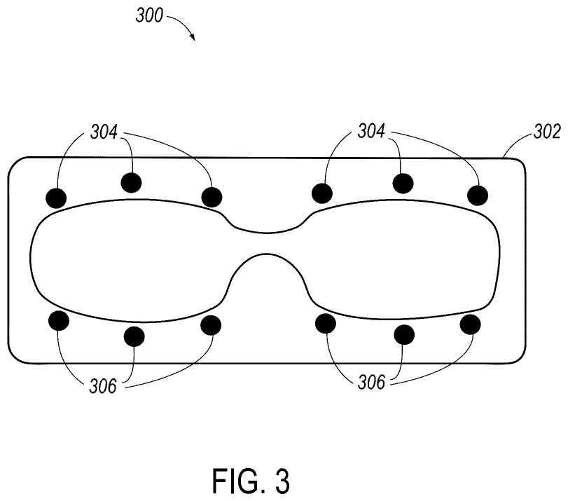

The frame 102 is designed to be worn over the eyes of a wearer, similar to a pair of goggles. In various embodiments, the frame 102 is substantially rigid, permitting minimal or no flexure under normal use conditions. In some embodiments, the flexure or deflection of the frame 102 at any point, while the OSD 100 is being worn, may be less than 5 mm compared to when the OSD 100 is not being worn. The frame 102 includes a skin engaging surface 104, illustrated more clearly in FIGS. 2A, 2B, and 3. The skin engaging surface 104 may be contoured to closely conform to the natural shape of the forehead and cheek bones. Although FIG. 1 illustrates a simple curve of the skin engaging surface 104, it should be appreciated that more complex shapes are also envisioned.

FIG. 1 illustrates the skin engaging surface 104 as being an integral part of the frame 102. However, in various other embodiments, the skin engaging surface 104 may be in the form of a compressible gasket. The compressible gasket may enable the frame 102 to fit snugly against the face of the wearer when the OSD 100 is worn. Such a compressible gasket may include, without limitation, a foam gasket, a rubber gasket, or a composite gasket. In yet other embodiments, the skin engaging surface 104 may be in the form of a flexible sub-frame, that is fixedly coupled to the frame 102 through springs. Any known type of springs may be used—for example, coil springs, flat springs, and so forth.

The frame 102 includes end pieces 106. The end pieces 106 may be an integral part of the frame 102. The end pieces 106 may simply be the lateral extremities of the frame. The end pieces 106 may include one or more fasteners (not illustrated) for coupling the strap element 108 to the frame 102. The fasteners may be any suitable type including, but not limited to, screws, bolts, press fit connectors, and so forth. Such removable fasteners may aid in replacing the strap element 108, for example, for repairing worn out or broken strap elements 108, or fitting a different sized strap element 108.

The strap element 108 is a head strap for securing the OSD 100 to the wearer's head, during use. The strap element 108 is designed to be in tension, such that the strap element 108 aids in pulling the frame 102 to fit snugly against the face of the wearer. In some embodiments, the strap element 108 may be an elastic strap, such as a natural rubber elastic band, a synthetic rubber elastic band, a polyester elastic fabric, a polyamide elastic fabric, or fabrics containing a portion of polyether-polyurea copolymer fibers (such as, Lycra™, Spandex™, or elastane). Other fabric blends, or fabric and rubber blends are also envisioned as suitable materials for the strap element 108.

In some alternate embodiments, the strap element 108 may be substantially inextensible. In such embodiments, the strap element 108 may be coupled to springs, which provide the required tensile force for snug fitting the frame 102 against the face of the wearer. Any known spring elements and mechanisms may be used.

In yet other alternate embodiments, the strap element 108 may further include an adjustment mechanism 110. The adjustment mechanism 110 is configured to adjust the length of the strap element 108. Consequently, the tension in the strap element 108 is adjusted during use, so as to snugly fit the frame 102 against the face of the wearer. In one embodiment, the adjustment mechanism 110 may include holes and a snap fit connector. In an alternate embodiment, the adjustment mechanism 110 may include a ratchet mechanism which helps tighten the strap simply by pulling. In such an implementation, the adjustment mechanism 110 may also include a ratchet release, to release or loosen the strap, thus facilitating ease of wearing and removal of the OSD 100. In yet another alternate embodiment, the adjustment mechanism 110 may include a spring mechanism that is configured to be extensible for ease of wear and removal of the OSD 100, and yet exert the required tension to the strap element 108 during use, so as to fit the frame 102 snugly against the face of the wearer. There are a number of other strap length or strap tension adjustment mechanisms known in the art. The present disclosure envisions all such known adjustment mechanisms as suitable alternatives to the adjustment mechanism 108 described herein.

For the optimal strap length and strap tension adjustment, the frame 102 may be substantially rigid, such that the frame 102 permits minimal to no flexure under normal use conditions. In addition to or alternative to the strap element 108 and adjustment mechanism 110 being configured to adjust the strap length or the strap tension, the frame 102 may also be configured to aid in the snug fit of the frame 102 against the face of the wearer. In one embodiment, the frame 102 may be flexible such that the frame 102 may wrap around a face of the wearer when worn, under tension of the strap element 108. In such an embodiment, the flexibility of the frame 102 may be limited to a flexible nose bridge, with the frame 102 being substantially rigid in other portions.

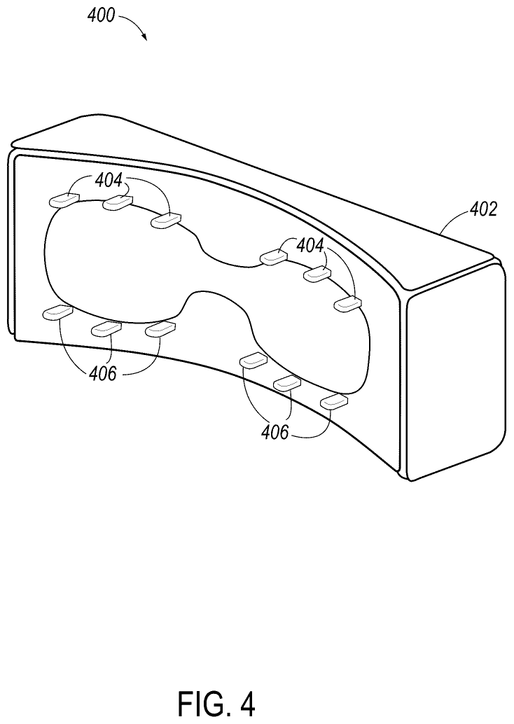

The frame 102 includes one or more electrodes (illustrated in FIG. 3) fixedly attached thereon. The electrodes provide electrical microcurrent stimulation to the orbital portions (illustrated in FIG. 4) of the eyes of the wearer. The tension of the strap element 108 is vital to a close fit of the frame 102 against the face of the wearer. This is particularly important for optimal contact of the electrodes with the face of the wearer, to minimize contact resistance, and thus effectively administer electrical microcurrent stimulation therapy to the wearer. In some embodiments the skin engaging surface of the electrodes is movable with respect to the frame 102. In some embodiments, the electrodes may be spring-loaded elements fixedly attached to the frame 102. The spring-loaded elements of each of the electrodes are configured to independently extend and/or retract, independent of other ones of the electrodes. In other embodiments, a compressible element such as a piece of compressible foam, or a spring may be disposed between and fixedly attached to the frame 102 and each one of the electrodes. In the aforementioned implementations, the electrodes may function as self-adjusting and therefore may provide the optimal contact pressure against the skin of the wearer. In embodiments where the skin engaging face 104 of the frame includes a compressible gasket, the electrodes may be fixedly attached to the compressible gasket. In other words, the compressible gasket may be viewed as being disposed between the frame 102 and the electrodes.

The frame 102 also includes one or more light emitters (illustrated in FIGS. 2A and 2B), fixedly attached thereon. In particular, the light emitters provide photo biomodulation stimulation therapy for the eyes. The light emitters may be configured to emit non-coherent low intensity light, having wavelengths in the range of 560 nm and 900 nm. More particularly, the light emitters may be configured to emit light in distinct wavelength bands, including at least two of a 560-600 nm wavelength band, a 620-670 nm wavelength band, and a 830-900 nm. These wavelength bands correspond to yellow light, red light, and near infrared (NIR) light respectively. In various embodiments, the light emitters may be configured to emit light having intensity peaks at at least two of 590 nm, 660 nm, and 850 nm wavelengths. In one embodiment, the light emitters emit light in all three wavelengths bands, and/or having peak intensity at all three wavelengths specified above. In various embodiments, the intensity of the light emitted at each wavelength is such that the light energy impinging on the cornea is between 30 mW/cm2 and 50 mW/cm2. In one exemplary embodiment, the light energy impinging on the cornea is 40 mW/cm2.

In some embodiments, the light emitters are fixedly attached to the eyepieces 112. The frame 102 includes a pair of eyepieces 112. The eyepieces 112 may be clear or at least partially see through. The eyepieces 112 are configured to allow the wearer to view their environment, while using the OSD 100 for therapy. In various embodiments, the eyepieces 112 may be made of a durable material with high optical clarity, for example, but not limited to, polycarbonate, acrylic, glass, and so forth. While FIG. 1 illustrates two distinct eyepieces 112, one for each eye, in some embodiments, OSD 100 may include a single eyepiece 112, shaped as a goggle, to cover both eyes.

In some embodiments, the eyepieces 112 may also include integrated optical elements to aim, and/or focus, and/or guide the light emitted by the light emitters towards the eye of the wearer. Alternatively, the optical elements to aim, and/or focus, and/or guide the light may be arranged within the frame 102, separate from the eyepieces 112.

In yet other embodiments, the OSD 100 may entirely omit the eyepieces 112, and the frame 102 may have an opaque front.

FIG. 1 indicates the direction of rear view of the OSD 100, along the direction R. FIGS. 2A and 2B illustrate the corresponding rear views of OSDs 200A and 200B, respectively, according to exemplary embodiments. FIGS. 2A and 2B illustrate the arrangement of light emitters. The light emitters provide photo biomodulation stimulation therapy for the eyes. The light emitters may be configured to emit non-coherent low intensity light, having wavelengths in the range of 560 nm and 900 nm. More particularly, the light emitters may be configured to emit light in distinct wavelength bands, including at least two of a 560-600 nm wavelength band, a 620-670 nm wavelength band, and a 830-900 nm. These wavelength bands correspond to yellow light, red light, and near infrared (NIR) light respectively. In various embodiments, the light emitters may be configured to emit light having intensity peaks at at least two of 590 nm, 660 nm, and 850 nm wavelengths. In one embodiment, the light emitters emit light in all three wavelengths bands, and/or having peak intensity at all three wavelengths specified above. In various embodiments, the intensity of the light emitted at each wavelength is such that the light energy impinging on the cornea is between 30 mW/cm2 and 50 mW/cm2. In one exemplary embodiment, the light energy impinging on the cornea is 40 mW/cm2.

FIG. 2A illustrates a rear view of an OSD 200A, according to one exemplary embodiment. OSD 200A includes frame 202A, and light emitters disposed in the shaded region 204. In various embodiments, the light emitters are disposed in an array, in an alternating fashion of different wavelengths. Such an arrangement may allow uniform illumination of the eye with all wavelengths. The region 204 is the periphery of the eyepiece 212. The light emitters may be fixedly coupled to the frame 202A. Alternatively, the light emitters may be fixedly coupled to the eyepiece 212. The eyepiece 212 may also include integrated optical elements (not illustrated) to aim, and/or focus, and/or guide, the light towards the eye of the wearer. Such optical elements may include lenses, Fresnel lenses, prism elements, optical filters, and so forth.

FIG. 2B illustrates a rear view of an OSD 200B, according to one exemplary embodiment. OSD 200B includes frame 202B, and light emitters disposed in the shaded region 206. In various embodiments, the light emitters are disposed in an array, in an alternating fashion of different wavelengths. Such an arrangement may allow uniform illumination of the eye with all wavelengths. The region 206 is the at the middle of the eyepiece 212. The light emitters may be fixedly coupled to the eyepiece 212. The eyepiece 212 may also include integrated optical elements (not illustrated) to aim, and/or focus, and/or guide, the light towards the eye of the wearer. Such optical elements may include lenses, Fresnel lenses, prism elements, optical filters, and so forth. In embodiments where the eyepiece 212 is omitted, and the frame 202B includes an opaque front, the light emitters may be fixedly coupled to the frame 202B.

While the light emitters are described as being arranged in the regions 204 and 206, any suitable arrangement of the light emitters is envisioned herein, without limitation. For example, the light emitters may be arranged in a matrix array, a linear array, a pentile array, a hexagon array, and so forth.

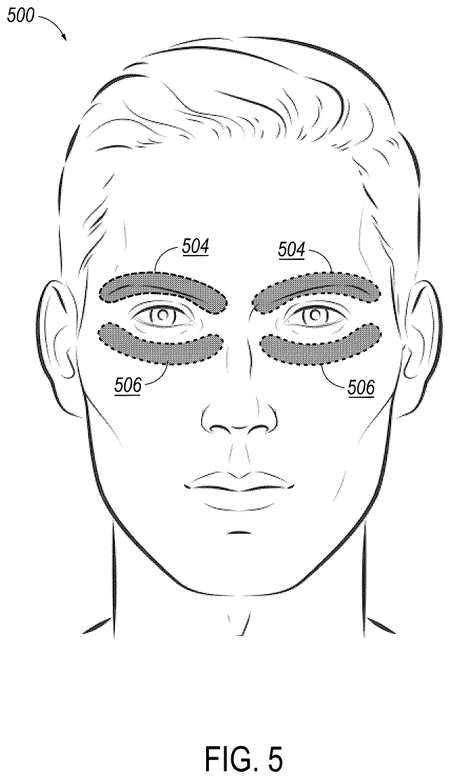

FIG. 3 illustrates a rear view of an OSD 300, according to one exemplary embodiment. The OSD 300 includes a frame 302, upper orbital electrodes 304, and lower orbital electrodes 306. The upper orbital electrodes 304 and lower orbital electrodes 306 are configured to provide electrical microcurrent stimulation to the orbital portions (illustrated in FIG. 5) of the eyes of the wearer. The microcurrent stimulation may be in the form of a pulsed waveform. In microcurrent stimulation may be in the form of frequency sweep pattern, for example, varying in the range of 1 Hz to 100 Hz. The intensity of the microcurrent stimulation may be, for example, between 50 μA and 200 μA.

The upper and lower orbital electrodes 304 and 306 are arranged to engage the wearer on an upper arc of an orbital cavity, and a lower arc of the orbital cavity, respectively. While FIG. 3 illustrates three electrodes for each of the upper and lower orbital positions, for each of the eye, other number of electrodes is also envisioned. For example, in one embodiment, each eye may only include one upper orbital electrode 304 and one lower orbital electrode 306, arranged in a diagonal position, or arranged at the upper and lower center positions. In another embodiment, each eye may have two upper orbital electrodes 304, and two lower orbital electrodes 306. In yet other embodiments, four or more pairs of electrodes are also envisioned. Electrodes 304 and 306 arranged in pairs allows for placement of at least one active electrode and at least one return electrode around the orbital positions of the wearer's eye. In some embodiments, active electrodes and return electrodes may be arranged in an alternating manner. In some other embodiments, active electrodes may be placed on the upper orbital arc, and the return electrodes on the lower orbital arc. In yet other embodiments, active electrodes may be placed on the lower orbital arc, and the return electrodes on the upper orbital arc.

The electrodes 304 and 306 may be made from any suitable material having high conductivity and high corrosion resistance. For example, the electrodes 304 and 306 may be made from stainless steel, nickel plated copper, gold plated copper, and so forth.

The electrodes 304 and 306 may be fixedly coupled to the frame 302. The electrodes provide electrical microcurrent stimulation to the orbital portions (illustrated in FIG. 5) of the eyes of the wearer. As described previously, in conjunction with FIG. 1, the tension of the strap element is vital to a close fit of the frame 302 against the face of the wearer. This is particularly important for optimal contact of the electrodes 304 and 306 with the face of the wearer, to minimize contact resistance, and thus effectively administer electrical microcurrent stimulation therapy to the wearer. In some embodiments the skin engaging surface of the electrodes 304 and 306 is movable with respect to the frame 302. In some embodiments, the electrodes 304 and 306 may be spring-loaded elements fixedly attached to the frame 302. The spring-loaded elements of each of the electrodes 304 and 306 are configured to independently extend and/or retract, independent of other ones of the electrodes 304 and 306. In some other embodiments, a compressible element such as a piece of compressible foam, or a spring may be disposed between and fixedly attached to the frame 302 and each of the individual electrodes 304 and 306. In aforementioned implementations, the electrodes 304 and 306 may function as self-adjusting and therefore may provide the optimal contact pressure against the skin of the wearer. In embodiments where the skin engaging face of the frame 302 includes a compressible gasket, the electrodes 304 and 306 may be fixedly attached to the compressible gasket. In other words, the compressible gasket may be viewed as being disposed between the frame 302 and the electrodes 304 and 306.

FIG. 4 illustrates a rear view of an OSD 400, according to one exemplary embodiment. The OSD 300 includes a frame 402, upper orbital electrodes 404, and lower orbital electrodes 406. The upper orbital electrodes 404 and lower orbital electrodes 406 are configured to provide electrical microcurrent stimulation to the orbital portions (illustrated in FIG. 5) of the eyes of the wearer.

The upper and lower orbital electrodes 404 and 406 are arranged to engage the wearer on an upper arc of an orbital cavity, and a lower arc of the orbital cavity, respectively. While FIG. 4 illustrates three electrodes for each of the upper and lower orbital positions, for each of the eye, other number of electrodes is also envisioned. For example, in one embodiment, each eye may only include one upper orbital electrode 404 and one lower orbital electrode 406, arranged in a diagonal position, or arranged at the upper and lower center positions. In another embodiment, each eye may have two upper orbital electrodes 404, and two lower orbital electrodes 406. In yet other embodiments, four or more pairs of electrodes are also envisioned. Electrodes 404 and 406 arranged in pairs allows for placement of at least one active electrode and at least one return electrode around the orbital positions of the wearer's eye. In some embodiments, active electrodes and return electrodes may be arranged in an alternating manner. In some other embodiments, active electrodes may be placed on the upper orbital arc, and the return electrodes on the lower orbital arc. In yet other embodiments, active electrodes may be placed on the lower orbital arc, and the return electrodes on the upper orbital arc.

The electrodes 404 and 406 may be made from any suitable material having high conductivity and high corrosion resistance. For example, the electrodes 404 and 406 may be made from stainless steel, nickel plated copper, gold plated copper, and so forth.

The electrodes 404 and 406 may be fixedly coupled to the frame 402. The electrodes provide electrical microcurrent stimulation to the orbital portions (illustrated in FIG. 5) of the eyes of the wearer. As described previously, in conjunction with FIG. 1, the tension of the strap element is vital to a close fit of the frame 402 against the face of the wearer. This is particularly important for optimal contact of the electrodes 404 and 406 with the face of the wearer, to minimize contact resistance, and thus effectively administer electrical microcurrent stimulation therapy to the wearer. In some embodiments the skin engaging surface of the electrodes 404 and 406 is movable with respect to the frame 402.

In some embodiments, the electrodes 404 and 406 may be spring-loaded elements fixedly attached to the frame 302. The spring-loaded elements of each of the electrodes 404 and 406 are configured to independently extend and/or retract, independent of other ones of the electrodes 404 and 406. The spring-loaded elements may be configured to provide a spring force in a direction perpendicular or transverse to an axis of the electrodes 404 and 406. Alternatively, the spring-loaded elements may be configured to provide a spring force in a direction along the axis of the electrodes 404 and 406. In yet another implementation, a spring-loaded element that provides a combination of axial and transverse spring force is also envisioned.

In yet other embodiments, the electrodes 404 and 406 may have include a flexible construction, wherein a body of the electrodes 404 and 406 exhibits flexure in a direction perpendicular or transverse to an axis of the electrodes 404 and 406. For example, the electrodes 404 and 406 may include a flexible stem and a conductive portion disposed at the skin engaging portion of the flexible stem. The flexible stem may be made of a suitable material which provides flexure during use, returns to its original shape and/or position while not in use, and resists permanent deformation due to repeated use. The flexible stem may be constructed from a polymer such as ABS, PVC, or PMMA. A conductive element may be disposed at the skin engaging portion of the electrodes 404 and 406.

In the aforementioned implementations, the electrodes 404 and 406 may function as self-adjusting and therefore may provide the optimal contact pressure against the skin of the wearer. In particular, implementations where the electrodes 404 and 406 exhibit transverse or perpendicular deflection, the electrodes 404 and 406 apply an optimal contact pressure at the natural recesses formed by the eye sockets.

FIG. 5 illustrates an electrode positioning 500 of the OSD, on the face of the wearer, according to various exemplary embodiments. The upper orbital electrodes 304, and 404 contact the wearer's skin in the upper orbital regions 504, while the lower orbital electrodes 306, and 406 contact the wearer's skin in the lower orbital regions 506. More particularly the upper orbital electrodes 304, and 404 are positioned to engage the superior eyelid crease above the eyeball, and the lower orbital electrodes 306, and 406 are positioned to engage the inferior eyelid crease below the eyeball. In other words, the upper orbital electrodes 304, and 404 and lower orbital electrodes 306, and 406 are positioned to engage the transpalpebral regions of the wearer's face. In various embodiments, the upper orbital electrodes 304, and 404 and lower orbital electrodes 306, and 406 are positioned to engage the wearer's face at the natural recess formed at the edges of the facial eye socket (orbit). As discussed above, in conjunction with FIG. 3, it is advantageous to have electrodes in pairs, so as to place at least one active electrode and at least one return electrode at the orbital positions around the wearer's eyes.

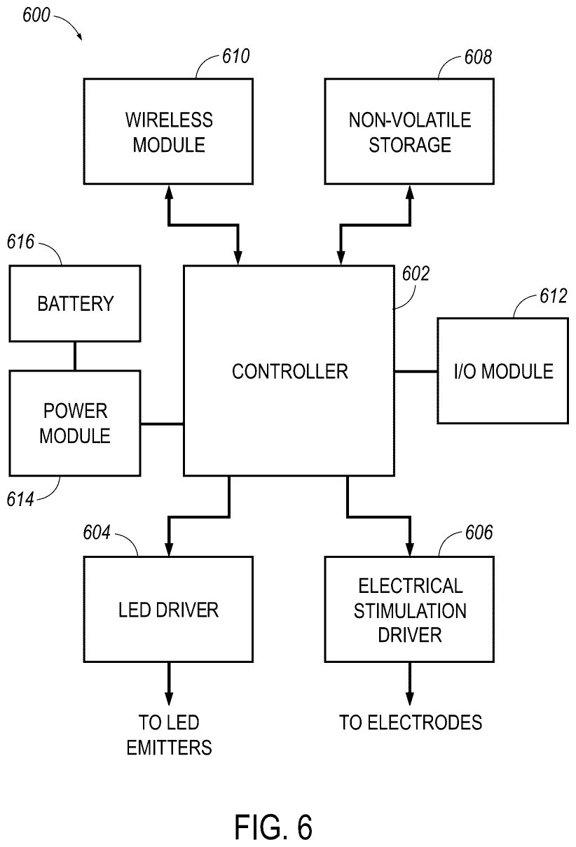

FIG. 6 illustrates a block diagram of an OSD control module 600, according to one exemplary embodiment. The OSD control module 600 includes a controller 602, an LED driver 604, an electrical stimulation driver 606, a non-volatile storage 608, a wireless module 610, and I/O module 612, a power module 614, and a battery 616. In various embodiments, the different blocks of the OSD control module 600 may be implemented on a single printed circuit board (PCB). In some embodiments, the power module 614, and battery 616 are distinct from the main PCB, so as to avoid electromagnetic interference caused by switching transients in the power module 614.

The controller 602 is configured to function as the central processing unit of the OSD control module 600. The controller 602 may be a microcontroller, or a microprocessor, or a mixed signal processor. The controller 602 is configured to provide low level drive signals to the LED driver 604, and the electrical stimulation driver 606. The controller 602 may control the LED driver 604 to vary parameters such as the wavelength of the light emission by switching the corresponding light emitters, the pulsing of the light emissions, the intensity of the light emissions, and the duration of the light emissions. The controller 602 may control the electrical stimulation driver 606 to control various parameters of microcurrent stimulation therapy, such as, but not limited to, the frequency of the microcurrent stimulation signal, the pulsing of the microcurrent stimulation signal, the amplitude of the microcurrent stimulation signal, and the duration of the microcurrent stimulation therapy.

The LED driver 604 is configured to drive the light emitters. In particular, the LED driver 604 is configured to convert the low level drive signals received from the controller 602, into power outputs that drive the light emitters. The LED driver 604 may be a constant voltage driver, or constant current driver, depending on the electrical connection of the light emitters.

The electrical stimulation driver 606 is configured to drive the microcurrent stimulation electrodes. In particular, the electrical stimulation driver 606 may be configured to convert the digital signals received from the controller 602, into analog signals to provide microcurrent stimulation therapy to the wearer, through the microcurrent stimulation electrodes.

The controller 602 may also store one or more therapy programs in the non-volatile storage 608. The therapy programs define the various parameters such as wavelength, frequency, pulsing, intensity, and duration of the therapy. The non-volatile storage 608 may also store program instructions necessary for the operation of the OSD controller module 600.

In some embodiments, the controller 602 may be configured to receive the therapy parameters from the wireless module 610. For example, the wireless module 610 may include an IEEE 802.11 Wi-Fi module, which may receive the therapy parameters from a local or remote system-such as a doctor's clinic or healthcare provider's office, or hospital. Alternatively, the wireless module 610 may include a short range local wireless communication radio, such as Bluetooth, or ZigBee, or NFC. Such a short range communication radio may be useful for configuring the OSD when the wearer (or patient) is in at the doctor's office for a consultation or follow-up.

In other embodiments, the controller 602 may be configured to receive the therapy parameters from the I/O module 612. The I/O module 612 may include input devices such as a touch interface, buttons, dials, a wired interface (such as USB, or serial port, and so forth). The input devices may be used to directly control the therapy parameters such as wavelength, frequency, pulsing, intensity, and duration of the therapy, and start and stop of a therapy session. This method may also be used to program the controller 602 with the therapy parameters, when the wearer (or patient) is in at the doctor's office for a consultation or follow-up. The I/O module 612 may also include output devices such as speakers, indicators, LCD displays, and so forth, to give feedback to the wearer during use or to the technician programming the OSD.

The power module 614 is configured to provide stable power to the other modules of the OSD control module 600. The power module 614 may include voltage regulators, upconverters and downconverters, battery charging circuitry, and battery protection circuitry.

The embodiments described herein are merely exemplary in nature. The features, elements or variations described herein may be used in any combination, to enable improved functionality over the products and solutions known in the art. The description of the embodiments presented herein shall not be considered limiting. Instead, the innovation is described in the appended claims, with implementation variations presented in the detailed description above.

1. An ophthalmic stimulation device comprising:
a frame;
a strap element coupled to end pieces of the frame;
a plurality of electrodes fixedly coupled to the frame, wherein the plurality of electrodes are positioned to engage at orbital positions around an eye of a wearer, and wherein the plurality of electrodes are configured to emit electrical microcurrents to stimulate the eye of the wearer;
a plurality of light emitters coupled to the frame, wherein the light emitters emit non-coherent light between 560 and 900 nm, and wherein the plurality of light emitters are configured to irradiate the eye of the wearer; and
a controller configured to control the operation of the plurality of electrodes and the plurality of light emitters.

2. The ophthalmic stimulation device of claim 1, wherein the frame is substantially rigid.

3. The ophthalmic stimulation device of claim 1, wherein the frame is flexible such that the frame may wrap around a face of the wearer when worn, under tension of the strap element.

4. The ophthalmic stimulation device of claim 1, wherein the strap element is an elastic band.

5. The ophthalmic stimulation device of claim 1, further comprising an adjustment mechanism to adjust the length of the strap element.

6. The ophthalmic stimulation device of claim 1, wherein the plurality of light emitters emit non-coherent light in at least two of a 560-600 nm wavelength band, a 620-670 nm wavelength band, and a 830-900 nm wavelength band.

7. The ophthalmic stimulation device of claim 1, wherein the plurality of light emitters emit non-coherent light having intensity peaks at at least two of 590 nm, 660 nm, and 850 nm wavelengths.

8. The ophthalmic stimulation device of claim 1, further comprising optical elements configured to at least one of aim, focus, and guide the non-coherent light emitted by the plurality of light emitters towards the eye of the wearer.

9. The ophthalmic stimulation device of claim 1, wherein the electrical microcurrent is administered as a pulsed waveform, comprising a frequency sweep pattern varying from 1 Hz to 100 Hz, and wherein the amplitude of the electrical microcurrent is between 50 μA and 200 μA.

10. The ophthalmic stimulation device of claim 1, wherein the plurality of electrodes are positioned to engage the wearer on an upper arc of an orbital cavity, a lower arc of the orbital cavity, or both.

11. The ophthalmic stimulation device of claim 1, wherein a skin engaging surface of the plurality of electrodes is movable with respect to the frame.

12. The ophthalmic stimulation device of claim 11, wherein the skin engaging surface of one of the plurality of electrodes is movable independently of the skin engaging surface of other ones of the plurality of electrodes.

13. The ophthalmic stimulation device of claim 11, wherein the compressible element is one of a compressible foam element, and a spring element.

14. The ophthalmic stimulation device of claim 1, further comprising a compressible element disposed between the frame and the plurality of electrodes, wherein the compressible element is fixedly attached to the frame and fixedly attached to the plurality of electrodes.

15. The ophthalmic stimulation device of claim 1, further comprising a plurality of compressible elements disposed between the frame and a corresponding one of the plurality of electrodes, wherein each of the plurality of compressible elements is fixedly attached to a corresponding one of the plurality of electrodes, and the frame.

16. The ophthalmic stimulation device of claim 1, wherein the controller is configured to control one or more of:
wavelength of light emission;
intensity of light emission;
pulsing of light emission;
duration of light emission;
frequency of electrical stimulation signal;
amplitude of electrical stimulation signal;
pulsing of electrical stimulation signal; and
duration of electrical stimulation.

17. The ophthalmic stimulation device of claim 1, wherein the controller is further configured to:
receive one or more treatment parameters;
store the treatment parameters on a non-volatile memory; and
control the operation of the plurality of electrodes and the plurality of light emitters based on the treatment parameters.

18. An ophthalmic stimulation device comprising:
a frame;
a strap element coupled to end pieces of the frame;
an eyepiece coupled to the frame;
a plurality of electrodes fixedly coupled to the frame, wherein the plurality of electrodes are positioned to engage at orbital positions around an eye of a wearer, and wherein the plurality of electrodes are configured to emit electrical microcurrents to stimulate the eye of the wearer;
a plurality of light emitters coupled to the eyepiece, wherein the light emitters emit non-coherent light between 560 and 900 nm, and wherein the plurality of light emitters are configured to irradiate the eye of the wearer; and
a controller configured to control the operation of the plurality of electrodes and the plurality of light emitters.

19. The ophthalmic stimulation device of claim 18, wherein the eyepiece further comprises optical elements to at least one of aim, focus, and guide the non-coherent light emitted by the plurality of light emitters towards the eye of the wearer.
